(19) United States
(12) Patent Application Publication
(10) Pub. No.: US 2013/0189770 A1
(43) Pub. Date: Jul. 25, 2013

(54) SAMPLE TESTING DEVICE

(71) Applicant: Aemtek, Inc., Fremont, CA (US)

(72) Inventors: Florence Q. Wu, Milpitas, CA (US); Ernest J. Oliveras, JR., Albany, CA (US); Yong-qing Huang, Newark, CA (US)

(73) Assignee: Aemtek, Inc., Fremont, CA (US)

(21) Appl. No.: 13/745,661

(22) Filed: Jan. 18, 2013

Related U.S. Application Data
(60) Provisional application No. 61/589,253, filed on Jan. 20, 2012.

(51) Int. Cl. C12Q 1/06

(52) U.S. Cl. CPC C12Q 1/06
USPC 435/288.4

(57) ABSTRACT

A sample holding device having a plurality of sample wells. A gutter surrounds the sample wells and is in fluidic communication with an overflow sample well. An upper surface of the device is sealed with a sealing film that fluidically isolates each sample well from each other and from the gutter and overflow well. The device is used to perform a most probable number (MPN) procedure.

### BACKGROUND OF THE INVENTION

This application claims the benefit of U.S. Provisional Application No. 61/589,253, filed on Jan. 20, 2012, the entirety of which is incorporated by reference herein.

The most probable number (MPN) is a procedure to estimate the population density of viable microorganisms in a test sample. It is based upon the application of the theory of probability to the numbers of observed positive growth responses to a standard dilution series of sample inoculum placed into a set number of culture media tubes. Positive growth response after incubation may be indicated by such observations as gas production in fermentation tubes, visible turbidity in broth tubes, color change of the liquid, or fluorescence when observed under UV lights, depending upon the type of media employed.

Generally, MPN is performed using several different sample holding containers of different volumes. For example, when implementing the 15-tube serial dilution method for MPN, 15 tubes need to be filled with sample fluid. Making serial dilutions and filling several individual containers is time consuming and increases risk of contamination, user error, and spillage. Devices, such as sample testing bags, exist that allow a user to only fill one container. However, these devices are specialized and require additional equipment.

### BRIEF SUMMARY OF THE INVENTION

Embodiments of the invention relate to a microbial enumeration device designed according to the MPN index and 95% confidence limits in the Standard Methods. The device can be used with a reagent to generate accurate, fast, and reliable assessment of total coliforms and E. coli in water sample. The device can have variable sized wells that mimic the statistical model of the traditional multiple tube fermentation method. In this manner, result reading is direct and MPN calculation is simple. It is accurate and sensitive and detects from one to up to 1600 MPN per 100 ml. The device is stand-alone sample holder and no expensive equipment is needed. The entire procedure involves a few steps and can be done in less than one minute per test.

The enumeration device can also be used with other reagent for assessment of MPN of microorganisms in liquid sample using the same statistical model and in a similar way.

One embodiment of the invention relates to a sample holding device having an upper portion having an upper surface, a bottom portion, a plurality of fluid wells opening at the upper surface; at least one overflow well opening at the upper surface, and a gutter at least partially surrounding the plurality of fluid wells, the gutter arranged to flow liquid into the at least one overflow well when the upper surface is horizontal.

In one aspect of the sample holding device, the plurality of fluid wells comprises five 10 ml wells, five 1 ml wells, and five 0.1 ml wells.

In another aspect, the total volume of the plurality of fluid wells and the at least one overflow well is not less than 100 ml.

In another aspect, the fluid wells are arranged in rows by size.

In another aspect, the plurality of fluid wells comprises 51 wells, with each well being no less than 1.96 ml and the 51st is for overflow liquid and connects to the surrounding gutter. The total volume of the plurality of fluid wells and the at least one overflow well is not less than 100 ml. This 51 well model generates maximum MPN number of 200.

In another aspect, the upper surface is configured to be film sealable around the gutter and between each fluid well by a planar sealing film such that each of the plurality of fluid wells is fluidically isolated from one another and from the at least one overflow fluid well and gutter.

In another aspect, the bottom portion is configured to mate with the upper portion such that the device is stackable with an identical sample holding device.

In another aspect, the upper portion and bottom portion are separated by a plurality of walls connected therebetween.

In another aspect, the upper portion is rectangular.

In another aspect, the gutter comprises one or more sloped troughs.

In another aspect, the sample holding device is constructed from a transparent material.

In another aspect, the transparent material can be polystyrene, polypropylene, or polycarbonate, polyethylene terephthalate (PETE).

Another embodiment of the invention relates to a method of using the device. The method includes obtaining the sample holding device and positioning the sample holding device such that the upper surface is horizontal. The sample liquid is poured over the plurality of wells to fill each well first and such that some of the sample liquid occupies the overflow well by spilling into the gutter.

In one aspect of the method, a sealing film is adhered to the upper surface to seal the sample liquid within the plurality of wells, the at least one overflow well, and gutter, such that each of the plurality of fluid wells are fluidically isolated from one another and from the at least one overflow fluid well and gutter.

In some embodiments and aspects, the sample holding device is developed based on the most probable number (MPN) principle. The most probable number method is also called multiple tube method and is based on statistical values of the results obtained. In the traditional method, measured sample volumes or of one or more dilutions are added to a series of tubes containing appropriate liquid medium. After appropriate incubation, tested organisms, if present, will grow and show characteristic change, such as color, turbidity, gas production or florescent, etc. The tubes are differentiated into having negative or positive results based on observation of the changes. The number and distribution of tubes showing a positive reaction will be used to estimate the most probable number of the tested organisms according to a MPN table.

In some embodiments and aspects, the sample holding device is used for estimating microbial density in liquid sample in terms of MPN value. The sample holding device can be arranged according to different models. The 16 well model is developed based on the 15-tube dilution method in the Standard Methods for the Examination of Water and Wastewater (20th edition, by American Public Health Association, American Water Works Association, and Water Environmental Federation). In this model, there are five 10 ml wells, five 1 ml wells, and five 0.1 ml wells. When filled full, each well contains 10 ml, 1 ml or 0.1 ml of sample. This various sized wells system replaces the need for making serial dilutions. After incubation, numbers of positive wells in each volume category will be counted and combination of the positives will be used to estimate MPN value and 95% confidence limits by referencing the MPN Index as shown in Table 9221.IV in the Standard Methods. The incorporation of the 16th well, i.e., the overflow well, is designed to capture the remaining volume in the 100 ml sample. If all wells including the 16th well show negative results, then the MPN value is determined to be less than 1 MPN per 100 ml.

The 51 wells model is developed based on the Standard Examination Methods for Drinking Water—Microbiology Parameters (People's Republic of China National Standards GB/T 5750.12-2006). In this model, there are 50 equal volume wells, each contains 1.96 ml sample when filled to maximum, and the 51st well is slightly larger to contain remain and excessive sample. After test setup and incubation, the numbers of positive wells will be counted and used to estimate MPN number according to the MPN value within 95% confidence limits according to Table 5 of the Chinese Standards GB/T 5750.12-2006. The 51st well is also the overflow well and along with the gutter, it is designed to capture the remaining volume in the 100 ml sample. If all wells including the 51st well show negative results, then the MPN value is determined to be less than 1 MPN per 100 ml.

### BRIEF DESCRIPTION OF THE DRAWINGS

FIG. 1 is a top view of a sample holding device, according to an embodiment of the invention.

FIG. 2 is a cross-sectional view along line A-A of FIG. 1.

FIGS. 3-5 are different perspective views of the sample holding device of FIG. 1.

FIG. 6 is a top perspective view of a sample holding device, according to an embodiment of the invention.

FIG. 7 is a bottom perspective view of the sample holding device of FIG. 6.

### DETAILED DESCRIPTION OF THE INVENTION

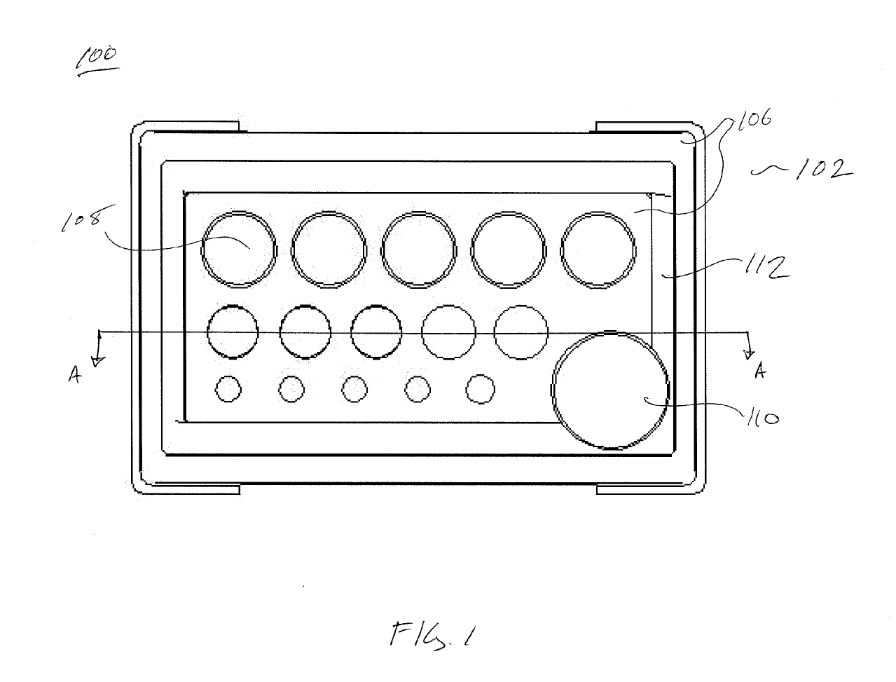

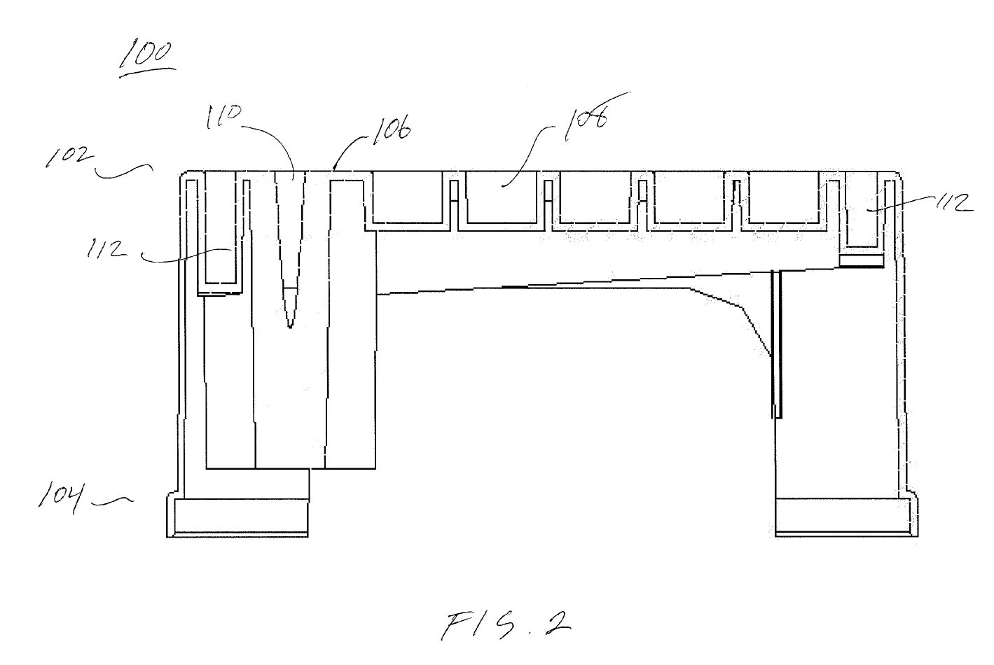

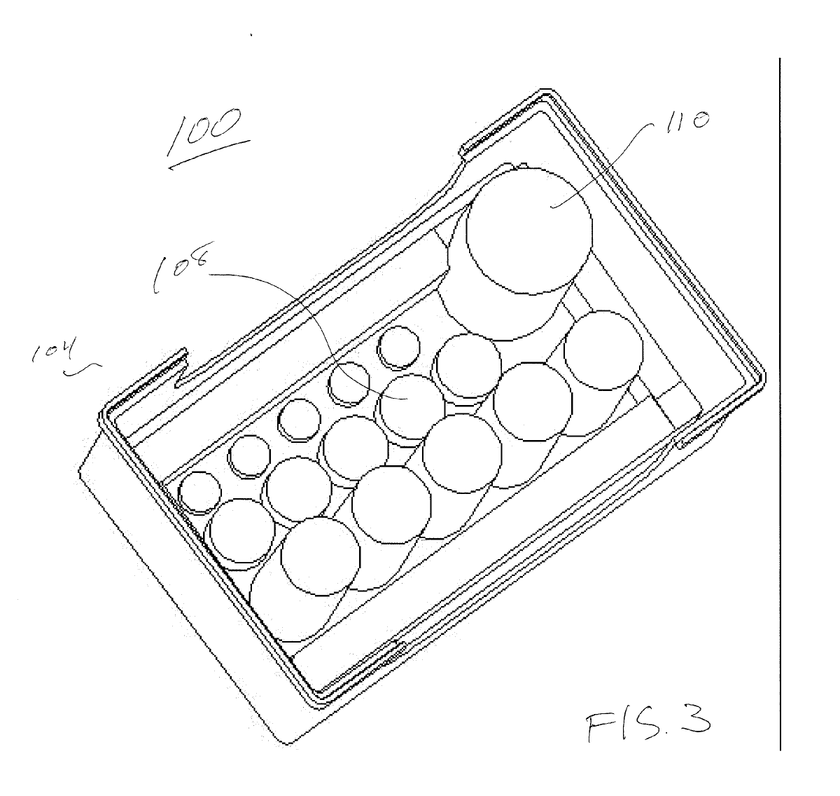

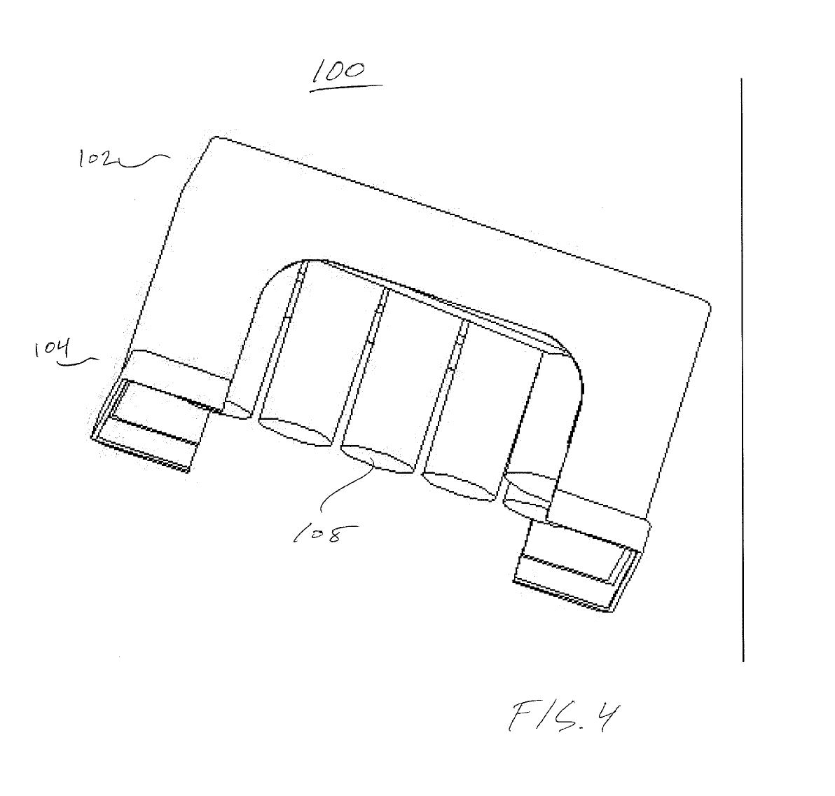

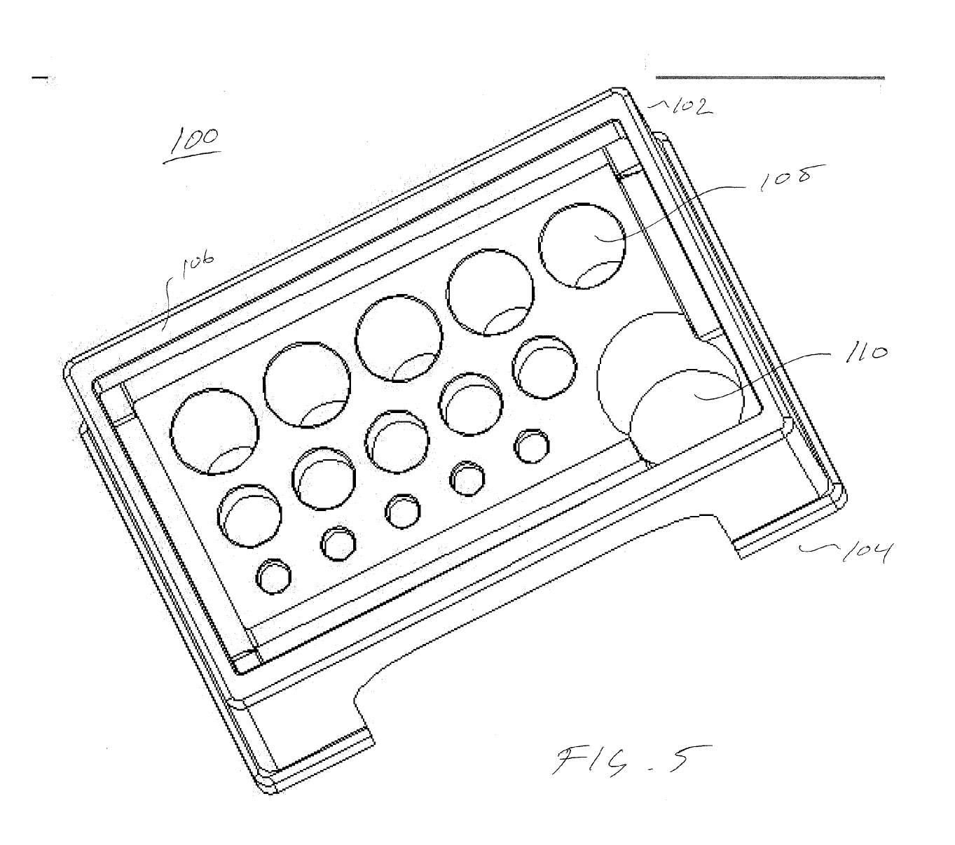

FIGS. 1-5 show views of a sample holding device (device) 100, according to an embodiment of the invention. The device 100 has a box-like shape including an upper portion 102 and a bottom portion 104. One or more walls connect the upper and lower portions. Portions of the walls can have cutouts, as shown, or the walls can be continuous. The bottom portion 104 can include stepped edges arranged to mate with edges of the upper portion. In this manner, the device can be stacked on top or beneath an identical device. The bottom portion 104 can have a footprint of a standard SBS microtiter plate.

The device 100 can be constructed from any material compatible with a desired testing procedure, e.g., a transparent non-fluorescing material. In some embodiments the device 100 is constructed from a clear material such as a polymer (e.g., polystyrene, polycarbonate, polypropylene) or glass. The device 100 can be constructed from a plurality of substructures or manufactured as on continuous piece of material, for example, a molded polymer.

The upper portion 102 of the device 100 is rectangular in shape about its outer extremities. The upper portion 102 is defined in part by an upper surface 106. The upper surface 106 is planar and is broken by a plurality of sample wells 108 that extend inside the device towards the bottom portion. The sample wells 108 are shown arranged in rows, however this is not required. The total capacity of the device 100 is not less than 100 ml in some embodiments. As shown, the device 100 includes five 10 ml wells, five 1 ml wells, five 0.1 ml wells, and at least one overflow well 110 that can hold at least 55 ml of fluid. This arrangement enables practice of the multiple tube fermentation method and its related statistical model for MPN. As shown, the device 100 enables performance of the 15-tube dilution method in the Standard Methods for the Examination of Water and Wastewater, described above. Another embodiment includes an arrangement of no less than 26 wells with variable volume to increase the maximum MPN value that can be detected.

The upper surface 106 also includes a gutter 112 that at least partially surrounds each well. As shown, a four sided gutter 112 includes sloped troughs that surround the sample wells 108, and is in fluidic communication with the overflow well 110. The gutter 112 has bottom surfaces that are non-parallel to the upper surface 106, i.e., each bottom surface slopes downwardly towards the bottom portion. The gutter 112 is also arranged in a stepped fashion such that the lowest point of the gutter ends at the overflow well 110. Accordingly, fluid that spills into the gutter 112 will flow into the overflow well 110 when the upper surface 106 is horizontal.

The upper surface 106 includes enough planar surface area between the samples wells 108, overflow well, and surrounding the gutter 112, such that when a planar sealing film is adhered to the upper surface, each sample well is fluidically isolated from one another. Similarly, the gutter 112 and overflow well 110 are also fluidically isolated from the sample wells 108.

In use, the device 100 can be used to perform the known multiple tube fermentation method and its statistical mode for MPN, instead of having multiple tubes and performing the time-consuming and labor-intensive traditional method. After being mixed with suitable reagent, such as Colitag™, the sample fluid can be poured directly into each of the sample wells 108 to fill them, and the remaining fluid is poured into the overflow well 110. When pouring the sample water, one does not need to take utmost care in filling the sample wells, since sample fluid poured onto portions of the upper surface 106 between the sample wells 108 will flow into the gutter and subsequently to the overflow well 110. A sealing film can then be applied to seal the device 100 via heat and/or pressure, and the sample is incubated. The sealing film can be transparent, semi-transparent, or completely opaque. After incubation, the number of positive wells is counted and used to obtain MPN value from the standard MPN table.

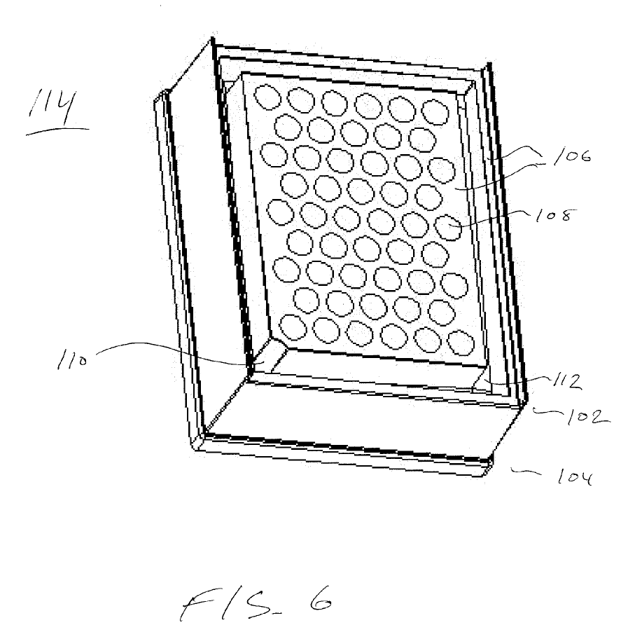

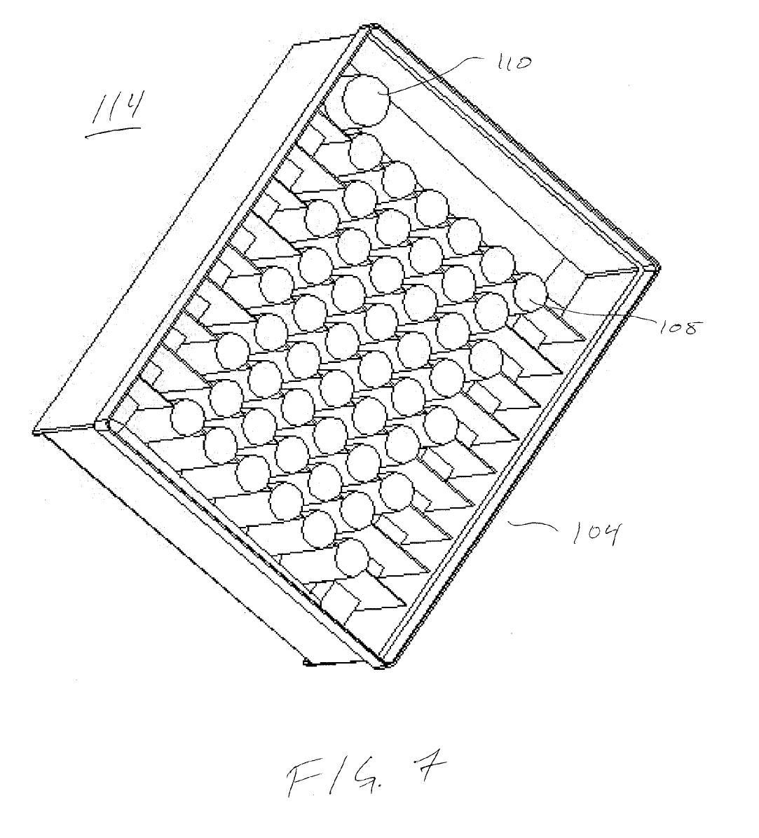

FIGS. 6 and 7 show views of a sample holding (device) 114, according to an embodiment of the invention. The device 114 is constructed similarly to the device 100 of FIGS. 1-5, and accordingly shares the same inventive features. However, device 114 differs by having an arrangement of 51 wells, each being no less than 1.96 ml, instead of the 16 wells (including overflow well 110) of device 100. The 51^{st} well is the overflow well 110, which is in fluidic communication with the gutter 112. The device 114 enables performance of The People's Republic of China National Standards GB/T 5750.12-2006, described above.

While the exemplary embodiments have been described in some detail for clarity of understanding and by way of example, a number of modifications, changes, and adaptations may be implemented and/or will be obvious to those as skilled in the art.

What is claimed is:

1. A sample holding device comprising:
an upper portion having an upper surface;
a bottom portion;
a plurality of fluid wells opening at the upper surface;
at least one overflow well opening at the upper surface; and
a gutter at least partially surrounding the plurality of fluid wells, the gutter arranged to flow liquid into the at least one overflow well when the upper surface is horizontal.

2. The device of claim 1, wherein the plurality of fluid wells comprises five 10 ml wells, five 1 ml wells, and five 0.1 ml wells.

3. The device of claim 2, wherein the total volume of the plurality of fluid wells and the at least one overflow well is not less than 100 ml.

4. The device of claim 2, wherein the fluid wells are arranged in rows by size.

5. The device of claim 1, wherein the plurality of fluid wells comprises 51 wells, with each well being no less than 1.96 ml.

6. The device of claim 1, wherein the upper surface is configured to be film sealable around the gutter and between each fluid well by a planar sealing film such that each of the plurality of fluid wells are fluidically isolated from one another and from the at least one overflow fluid well and gutter.

7. The device of claim 1, wherein the bottom portion is configured to mate with the upper portion such that the device is stackable with an identical sample holding device.

8. The device of claim 1, wherein the upper portion and bottom portion are separated by a plurality of walls connected therebetween.

9. The device of claim 1, wherein the upper portion is rectangular.

10. The device of claim 1, wherein the gutter comprises one or more sloped troughs.

11. The device of claim 1, wherein the sample holding device is constructed from a transparent material.

12. The device of claim 11, wherein the transparent material comprises polystyrene, polypropylene, polycarbonate, or polyethylene terephthalate (PETE).

13. A method comprising:
obtaining the sample holding device of claim 1 and positioning the sample holding device such that the upper surface is horizontal, and
pouring a sample liquid over the plurality of wells to fill each well and such that some of the sample liquid occupies the overflow well by spilling into the gutter.

14. The method of claim 13, further comprising adhering a sealing film to the upper surface to seal the sample liquid within the plurality of wells, the at least one overflow well, and gutter, such that each of the plurality of fluid wells are fluidically isolated from one another and from the at least one overflow fluid well and gutter.

15. The method of claim 13, wherein the sample holding device is used for most probable number (MPN) index and 95% confidence limits.

16. The method of claim 13, wherein the sample holding device is used for estimating microbial density in liquid sample in terms of MPN value.
